(19) United States
(12) Patent Application Publication
(10) Pub. No.: US 2014/0309597 A1
(43) Pub. Date: Oct. 16, 2014

(54) APPLICATOR FOR DELIVERING A SINGLE DOSE OF MEDICINE TO AN INNER CANAL OF AN EAR

(71) Applicants: Jeremiah Joseph Lowney, Milton, MA (US); Michael Barenboym, Cambridge, MA (US); Kenneth E. Olson, Foxboro, MA (US)

(72) Inventors: Jeremiah Joseph Lowney, Milton, MA (US); Michael Barenboym, Cambridge, MA (US); Kenneth E. Olson, Foxboro, MA (US)

(21) Appl. No.: 14/220,622

(22) Filed: Mar. 20, 2014

Related U.S. Application Data
(63) Continuation-in-part of application No. 13/651,774, filed on Oct. 15, 2012.

(51) Int. Cl. A61F 11/00; A61M 31/00

(52) U.S. Cl. CPC A61F 11/00; A61M 31/002
USPC 604/285

(57) ABSTRACT

An applicator for delivering a single dose of medicine into an inner canal of a person using a finger is disclosed. In one embodiment, the applicator includes a body having an open end and a closed end. A substantially solid compounded medicine is attached to the outer surface of the closed end of the body. The substantially solid compounded medicine melts to a liquid when disposed within the inner canal of the ear. A projection extends outwardly from the closed end of the body, with the compounded medicine surrounding the projection.

### RELATED APPLICATION DATA

This application is a continuation-in-part application of U.S. patent application Ser. No. 13/651,774, filed on Oct. 15, 2012, the entire contents of which is incorporated herein by reference in its entirety.

### FIELD

Aspects of this invention relate generally to an applicator for applying medicine to an ear, and in particular, to an applicator for applying a single dose of a compounded medication to an inner canal of an ear.

### BACKGROUND

Many medications and/or formulas for external ear ailments are in the form of liquid solution. Inserting drops into the ear of a person or an animal can be problematic. The person is asked to place a specific number of drops of the solution into one or each ear canal. After the drops are inserted into the ear canal the person is then instructed to lay on ones side for an approximate amount of time or put a cotton ball in the ear to attempt to maintain the solution in the inner canal of the ear for a predetermined amount of time. The application of drops into the ear with the above instructions has proven to be very difficult and time consuming. It is difficult for one to know exactly how many drops one is placing into the ear canal due to the awkward way one must tilt the head while squeezing the bottle of solution. There is a high degree of variability in the actual number of drops placed. Another problem with the current application of solution/drops into the ear canal is the need for a cotton ball. The two-step process of placing drops into the ear and then quickly inserting a cotton ball not only is cumbersome but the most common types of cotton balls are very absorbable and could potentially absorb much of the solution placed into the ear canal. Another problem with the current instructions for placing solution into the ear canal is asking one to lay on their side for a predetermine amount of time. The time spent on their side can vary, may decrease comfort level, and be an overall nuisance.

Other factors that make the current instructions for the application of drops into the ear very difficult can be when trying to treat infants, children, and animals. Parents can have an extremely agitated child who may also be in pain. Attempting to place ear drops accurately and then maintaining the drops in the ear canal can be almost impossible. Similar problems can be encountered when applying drops into the ear of an animal.

### SUMMARY

The present invention is an applicator for delivering a single dose of medicine into an inner canal of a person using a finger. In one embodiment, the applicator comprises a tubular body having an open end and a closed end. The open end is wound upon itself to form a rim positioned substantially adjacent and close to the closed end. The closed end has an inner surface and an outer surface. The applicator further comprises a substantially solid compounded medicine attached to the outer surface of the closed end of the tubular body. The substantially solid compounded medicine melts to a liquid when disposed within the inner canal of the ear. The rim is adapted to be disposed outside of the inner canal of the ear and the tubular body may be unrolled by pushing the finger against the inner surface of the closed end to a position where the closed end and the substantially solid compounded medicine are disposed within the inner canal of the ear.

In accordance with another aspect, an applicator for delivering a single dose of medicine into an inner canal of an ear using a finger includes a body having an open end and a closed end, with the closed end having an inner surface and an outer surface. A projection extends outwardly from the outer surface of the closed end, and a substantially solid compounded medicine surrounds the projection.

The body may taper from its open end toward its closed end. The projection may include a neck portion that extends directly outwardly from the outer surface of the body, and a second spherical portion that is connected to and extends directly outwardly from the neck portion.

In accordance with a further aspect, an applicator for delivering a single dose of medicine into an inner canal of an ear using a finger may include a body having an open end and a closed end and tapering from the open end toward the closed end. The closed end has an inner surface and an outer surface. A projection including a neck portion extends outwardly from the outer surface of the closed end and a spherical portion is connected to the neck portion. A substantially solid compounded medicine surrounds the projection.

Features and advantages of applicators for delivery medicine as disclosed here will be further understood from the following detailed disclosure of certain embodiments.

### BRIEF DESCRIPTION OF THE DRAWINGS

FIG. 1 is a perspective view of an applicator according to the present invention disposed with an inner canal of an ear of a person;

FIG. 2 is a perspective view of the applicator prior to insertion into the inner canal of the ear;

FIG. 3 is a side view of the applicator unrolled or deployed;

FIG. 4A is a side view of a cotton tube cut to a predetermined length from a roll of medical grade cotton tubing;

FIG. 4B is a perspective view of the cotton tube with an open end and a closed end formed by stitching;

FIG. 4C is a cross-section view the applicator with the open end rolled upon itself to form a rim and the closed end having inner and outer surfaces;

FIG. 4D is a side view of the applicator with the open end rolled upon itself to form a rim and a single dose of medicine applied or otherwise affixed to the outer surface of the closed end;

FIG. 4E is cross-section view of a container or package comprising a sufficient number of applicators (one dose per applicator) for a given prescription—such as twice a day for ten days or twenty applicators;

FIG. 5 is a perspective view of another embodiment of an applicator.

FIG. 6 is a perspective view of a further embodiment of an applicator.

FIG. 7 is an elevation view of the applicator of FIG. 6.

FIG. 8 is a perspective section view of the applicator of FIG. 6.

FIG. 9 is an elevation view of yet another embodiment of an applicator.

The figures referred to above are not drawn necessarily to scale, should be understood to provide a representation of particular embodiments, and are merely conceptual in nature and illustrative of the principles involved. Some features of the applicator depicted in the drawings have been enlarged or distorted relative to others to facilitate explanation and understanding. The same reference numbers are used in the drawings for similar or identical components and features shown in various alternative embodiments. Applicators as disclosed herein would have configurations and components determined, in part, by the intended application and environment in which they are used.

### DETAILED DESCRIPTION OF CERTAIN PREFERRED EMBODIMENTS

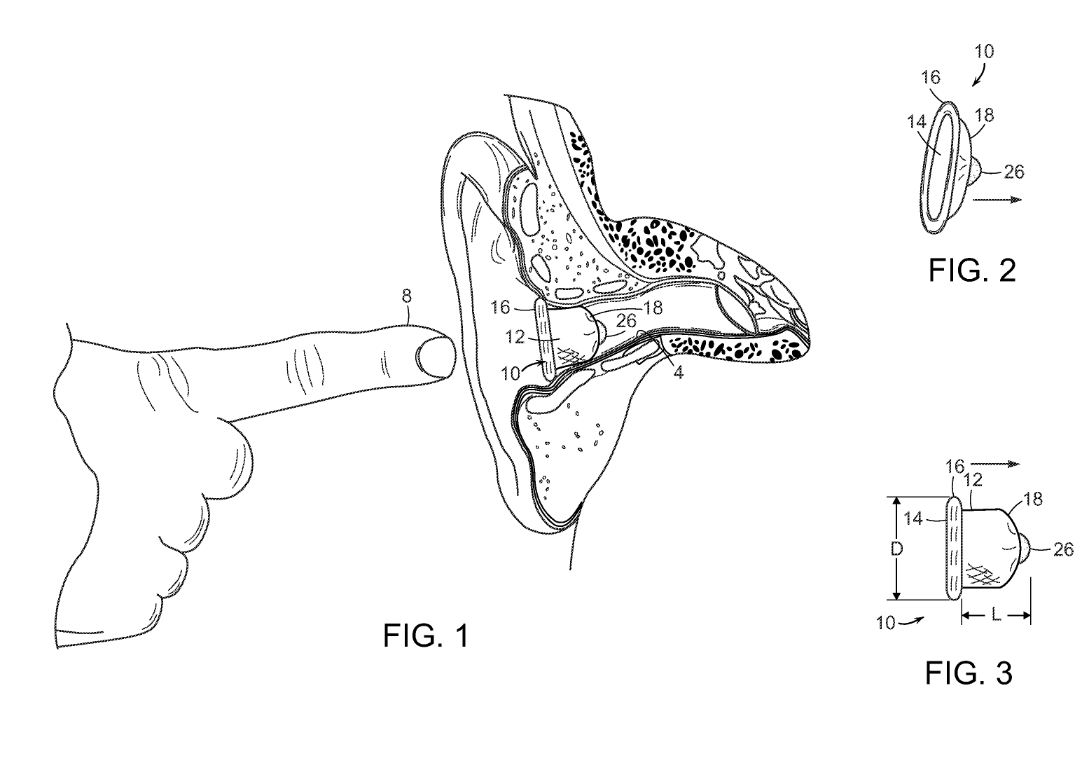

Referring to FIG. 1, where an applicator 10 according to the present invention is shown disposed within an inner canal 4 of an ear 2 of a person (not shown). Applicator 10 is designed to deliver a single dose of medicine to inner canal 4 of ear 2 using a finger 8. A person (not shown) may use his/her own finger to insert applicator 10 into his/her ear 2. Alternatively, a person (not shown) may use his/her finger to insert applicator 10 into the ear 2 of another person. Applicator 10 may also be used in connection with animals or any ear having an inner canal.

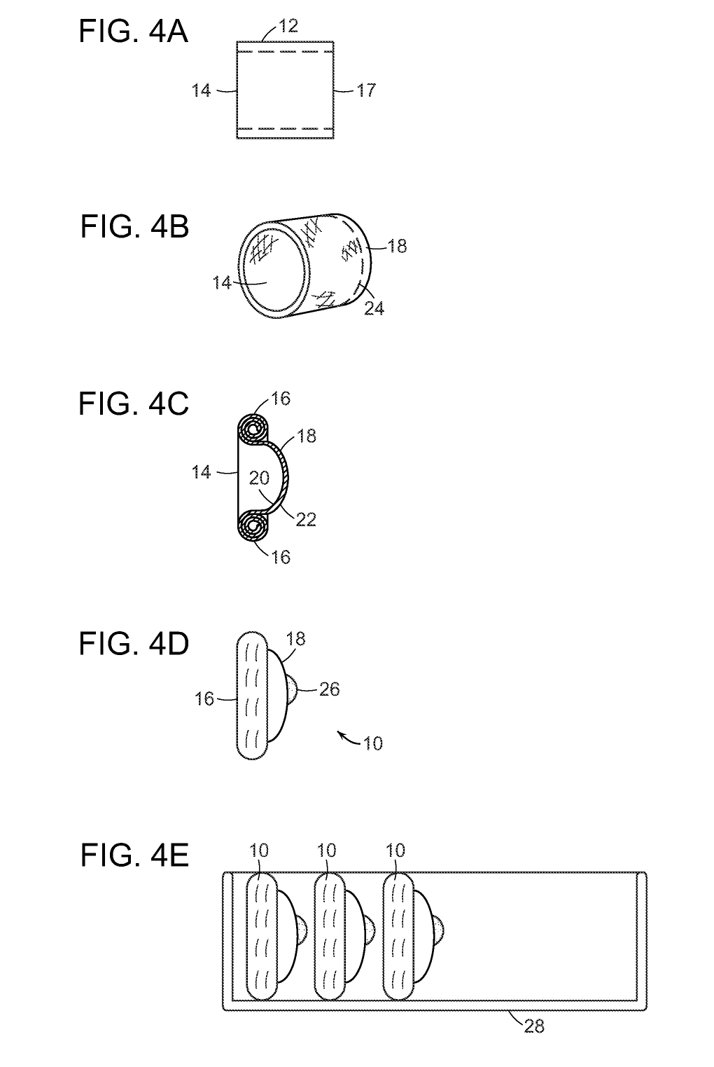

Referring to FIGS. 2-3, applicator 10 generally comprises a tubular body 12 having an open end 14 and a closed end 18. Open end 14 is wound upon itself to form a rim 16 positioned substantially adjacent and close to closed end 18. Applicator 10 further comprises a pre-determined amount of a substantially solid compounded medicine 26 attached or otherwise affixed to closed end 18 of tubular body 12. The substantially solid compounded medicine 26 melts to a liquid when disposed within inner canal 4 of ear 2. Rim 16 has a diameter slightly larger than the diameter of tubular body 12 and is disposed outside of inner canal 4. Tubular body 12 may be unrolled by pushing finger 8 against inner surface 20 (FIG. 4C) of closed end 18 to a position where outside surface 22 (FIG. 4C) of closed end 18 and the substantially solid compounded medicine 26 are disposed within inner canal 4. The length “L” that closed end 16 may be pushed outward from open end 14 or rim 16 by finger 8 depends upon the size of the person's finger 8 and ear 2 but in most cases would not exceed 0.50 inches for an adult.

Referring to FIG. 4A, tubular body 12 of applicator 10 is made from a roll of soft medical grade thin tubular woven cotton fabric which is widely available. Tubular body 12 is cut from the roll of cotton fabric having a diameter of about 0.75 inches and a length of about 0.75 inches to form open ends 14 and 17. These dimensions provide a tubular body 12 that can be properly formed and inserted in an inner canal of many adults. Larger dimensions may be needed for adults with a larger inner canal. Smaller dimensions may be needed for infants and children or in other uses such as ears of animals.

Referring to FIG. 4B, open end 17 is sewn closed by conventional stitching 18 to form closed end 18, and open end 14 is left open to allow insertion of finger 8.

Referring to FIG. 4C, starting from open end 14, the cotton fabric is tightly rolled toward closed end 18 similar to how a packaged condom is rolled. Closed end 18 has inner and outer surfaces 20 and 22. As will be more fully described herein, the finger 8 (FIG. 1) is pushed against inner surface 20 to push or unroll closed end 18 into inner canal 4 of ear 2 (FIG. 1).

Referring to FIG. 4D, a pre-determined amount of a substantially compounded solid medicine 26 is then applied to closed end 18 of tubular body 12. Medicine 26 has an active ingredient that is compounded with an agent such as glycerinated gelatin (name brand example of MBK Base) or cocoa butter (or another agent) and heated to liquid form that solidifies at room temperature with the active ingredient. Medicine 26 melts at body temperature or when placed within inner canal 4. In its liquid and heated form the, the compound is placed onto the center of closed end 18 and allowed to solidify as medicine 26 having a single dose of the active ingredient needed to treat the ear for a given condition. Depending upon the condition being treated, various types of active ingredients may be used. By way of example only, Otitis Externa may be treated with an antibiotic medication such as ciprofloxin as the active ingredient of medicine 26 which is formed by compounding a solution of the antibiotic mediation with glycerinated gelatin as an agent. The amount of active ingredient for a single dose will vary depending upon the condition be treated as is well known in the art.

Referring to FIG. 4E, applicator 10 may be sealed within a clear plastic or paper package individually (not shown) and/or placed in a multi-pack package or container 28. For an ear treatment with a pre-determined duration, multi-pack 28 can have a sealed full course of treatment. A treatment course of inserting applicator 10 into ear 2 three times a day for seven days would have twenty-one applicators in package 28. Applicator 10 can be stored at room temperature or refrigerated according to the active ingredient requirements. Applicator 10 may also be packaged as “as needed” for active ingredients such as, but not limited to, ear wax removers and ear ache relievers. Any active ingredient for ear 2 that can be compounded to a solid at room temperature and melt at body temperature can be placed into inner canal 4 with applicator 10. Compounding of active ingredients and agents are well known in the art.

In use, a person may remove applicator 10 from container 28 and place open end 14 on his or her finger 8. The person may then place rim 16 of applicator 10 against and outside of inner canal 4 of ear 2. Thereafter, the person may push his her finger against inner surface 20 thereby unrolling closed end 18 and medicine 26 into inner canal 4 of ear 2. The soft feature of tubular body 12 allows it to conform to the shape of inner canal thereby providing a seal so that medicine 26 when melted will remain within inner canal 2 and not drip outside of ear 2. Applicator 10 constitutes a major breakthrough in the insertion of active ingredients into inner canal 4 and feeling comfortable knowing one is receiving the accurate amount of medication and that it will remain in ear 2 comfortably for the proper duration.

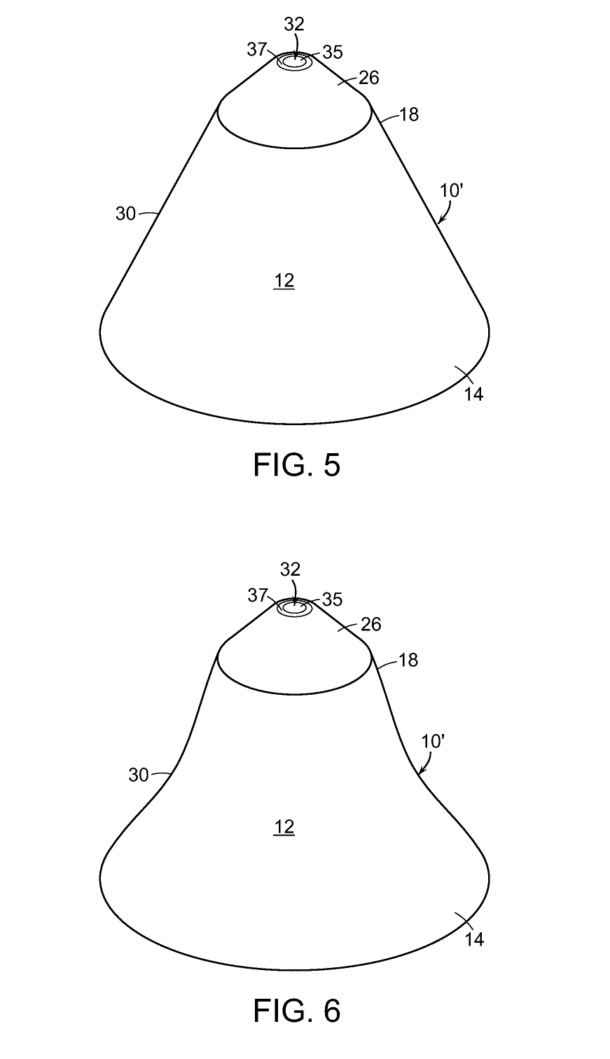

Another embodiment is shown in FIG. 5. In this embodiment, applicator 10′ is not configured to be wound or rolled up. Rather, applicator 10′ is designed to be substantially rigid. That is, applicator 10′ is designed to maintain its shape during use. Although the material used to form applicator 10′ may have some flexibility or resilience, applicator 10′ is designed and intended to substantially maintain its shape during use when applying the solid compounded medicine to the ear of the user.

In certain embodiments, applicator 10′ may be formed of an elastomer, or any other suitable rubber-like material, such as silicone. Other suitable materials for applicator 10′ will become readily apparent to those skilled in the art, given the benefit of this disclosure.

Applicator 10′ may have substantially the shape of a frustum of a cone, with open end 14 being wider than closed end 18. In other words, body 12 of applicator 10′ tapers from open end 14 toward closed end 18.

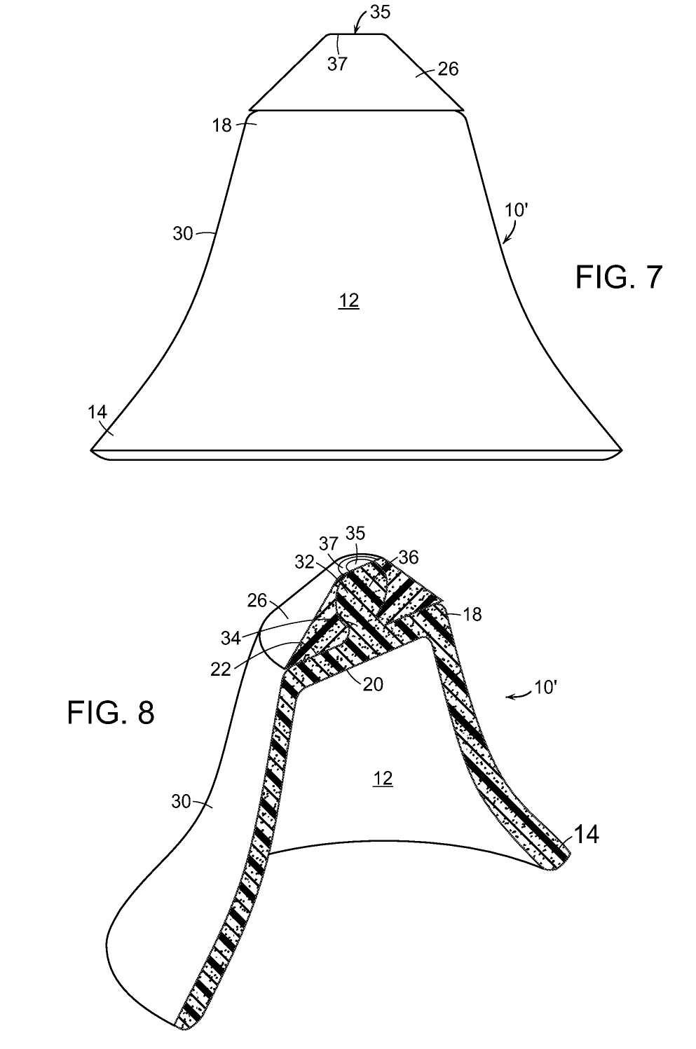

In certain embodiments, as shown in FIGS. 6-8, the wall 30 of applicator 10′ may flare outwardly as it approaches open end 14 of applicator 10′, thereby providing applicator 10′ with a bell shaped configuration.

A projection 32 is connected to and extends outwardly from outer surface 22 of closed end 18 of applicator 10′. Medicine 26 surrounds projection 32 such that projection 32 serves to retain medicine 26 on applicator 10′ before and during application of medicine 26 to the user's ear. It is to be appreciated that projection 32 can take any desired shape.

In the embodiment illustrated in FIG. 8, projection 32 includes a first or neck portion 34 that is connected to and extends directly outwardly from outer surface 22, and a second or spherical portion 36 that is connected to and extends directly outwardly from neck portion 34. In certain embodiments, projection 32 is of unitary, that is, one-piece, construction with body 12 of applicator 10′. In other embodiments, projection 32 may be a separate element secured to body 12 by an adhesive or any other suitable means of securing the elements together.

As seen in FIGS. 6-8, an upper endmost surface 35 of projection 32 is not covered by medicine 26 and is exposed to an exterior of applicator 10. In certain embodiments endmost surface 35 of projection 32 is substantially flush with an upper endmost surface 37 of medicine 26.

In the illustrated embodiment, compounded medicine 26 surrounding projection 32 of applicator 10′ has the shape of the frustum of a cone. However, it is to be appreciated that compounded medicine 26 is malleable, and can take on any desired shape.

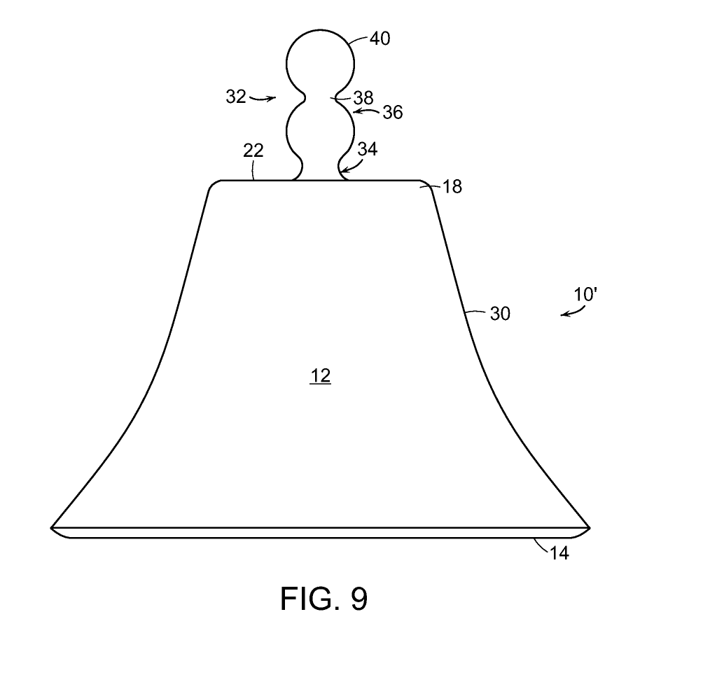

Another embodiment is shown in FIG. 9, in which a second neck portion 38 is connected to and extends directly outwardly from spherical portion 36, and a second spherical portion 40 is connected to and extends directly outwardly from second neck portion 38.

It is to be appreciated that the various portions of projection 32 can take any desired shape, and that other suitable shapes for the various portions of projection 32 will become readily apparent to those skilled in the art, given the benefit of this disclosure.

Thus, while there have been shown, described, and pointed out fundamental novel features of various embodiments, it will be understood that various omissions, substitutions, and changes in the form and details of the devices illustrated, and in their operation, may be made by those skilled in the art without departing from the spirit and scope of the invention. For example, it is expressly intended that all combinations of those elements and/or steps which perform substantially the same function, in substantially the same way, to achieve the same results are within the scope of the invention. Substitutions of elements from one described embodiment to another are also fully intended and contemplated. It is the intention, therefore, to be limited only as indicated by the scope of the claims appended hereto.

What is claimed is:

1. An applicator for delivering a single dose of medicine into an inner canal of an ear using a finger comprising:
a body having an open end and a closed end; the closed end having an inner surface and an outer surface;
a projection extending outwardly from the outer surface of the closed end; and
a substantially solid compounded medicine surrounding the projection.

2. The applicator of claim 1, wherein the body is substantially rigid.

3. The applicator of claim 1, wherein the body is formed of an elastomer.

4. The applicator of claim 1, wherein the body is formed of silicone.

5. The applicator of claim 1, wherein the body tapers from its open end toward its closed end.

6. The applicator of claim 1, wherein a wall of the body flares outwardly at the open end, providing the body with a bell shape.

7. The applicator of claim 1, wherein the projection includes a neck portion extending outwardly from the outer surface and a spherical portion connected to the neck portion.

8. The applicator of claim 1, wherein the projection is of unitary construction with the body.

9. The applicator of claim 1, wherein the substantially solid compounded medicine has an antibiotic medication as an active ingredient and glycerinated gelatin as an agent.

10. The applicator of claim 9, wherein the antibiotic medication is a solution of ciprofloxin.

11. The applicator of claim 1, wherein an upper endmost surface of the projection is exposed to an exterior of the applicator.

12. The applicator of claim 11, wherein the upper endmost surface of the projection is substantially flush with an upper endmost surface of the compounded medicine.

13. An applicator for delivering a single dose of medicine into an inner canal of an ear using a finger comprising:
a body having an open end and a closed end and tapering from the open end toward the closed end, the closed end having an inner surface and an outer surface;
a projection including a neck portion extending outwardly from the outer surface of the closed end and a spherical portion connected to the neck portion; and
a substantially solid compounded medicine surrounding the projection.

14. The applicator of claim 13, wherein the body is substantially rigid.

15. The applicator of claim 13, wherein a wall of the body flares outwardly at the open end, providing the body with a bell shape.

16. The applicator of claim 13, wherein the projection is of unitary construction with the body.

17. The applicator of claim 13, wherein the substantially solid compounded medicine has an antibiotic medication as an active ingredient and glycerinated gelatin as an agent.

18. The applicator of claim 17, wherein the antibiotic medication is a solution of ciprofloxin.

19. The applicator of claim 13, wherein an upper endmost surface of the projection is exposed to an exterior of the applicator.

20. The applicator of claim 19, wherein the upper endmost surface of the projection is substantially flush with an upper endmost surface of the compounded medicine.
